(19) United States
(12) Patent Application Publication
(10) Pub. No.: US 2025/0264320 A1
(43) Pub. Date: Aug. 21, 2025

(54) SET OF INDUCTOR COILS FOR NON-DESTRUCTIVE MEASUREMENT OF DEFORMATION IN PAVEMENT STRUCTURES AND METHOD OF USING THE SAME

(71) Applicants: PETRÓLEO BRASILEIRO S.A. - PETROBRAS, Rio de Janeiro (BR); UNIVERSIDADE FEDERAL DO RIO GRANDE DO SUL - UFRGS, Porto Alegre (BR)

(72) Inventors: MARGARETH CARVALHO COUTINHO CRAVO, Rio de Janeiro (BR); PAOLO BUCCIONI, Porto Alegre (BR); ANDREW ROBERT DAWSON, Nottingham (BR); DIONÍSIO DOERING, Menlo Park, CA (US); LÉLIO ANTÔNIO TEIXEIRA BRITO, Porto Alegre (BR); HENRIQUE FALCK GRIMM, Porto Alegre (BR); FABRICIO TRESCASTRO, Porto Alegre (BR); MATHEUS FERREIRA MATUELLA, Porto Alegre (BR); WASHINGTON PERES NÚÑES, Porto Alegre (BR); LUIS ALBERTO HERRMANN DO NASCIMENTO, Rio de Janeiro (BR)

(73) Assignees: PETRÓLEO BRASILEIRO S.A. - PETROBRAS, Rio de Janeiro (BR); UNIVERSIDADE FEDERAL DO RIO GRANDE DO SUL - UFRGS, Porto Alegre (BR)

(21) Appl. No.: 19/052,659

(22) Filed: Feb. 13, 2025

(30) Foreign Application Priority Data
Feb. 15, 2024 (BR) 1020240029550

(51) Int. Cl. G01B 7/14; G01N 27/82; G01N 33/38

(52) U.S. Cl. CPC G01B 7/14; G01N 27/82; G01N 33/38

(57) ABSTRACT

The invention consists of a system comprising an emitting PCB 10 and one or more receiving PCBs 10, each PCB comprising three coils. The one or more receiving PCBs 10 are positioned on pavement layers while the pavement is being prepared. After the pavement is complete, tests are performed to cause deformations in the pavement. The emitting PCB 10 is positioned on the pavement surface so as to form a pair with one of the receiving PCBs 10. A signal is emitted by a signal generator 20 to the emitting PCB 10, inducing a magnetic field in its coils, which in turn is captured by the coils of the receiving PCB 10 of the pair. The signal resulting in the receiving PCB 10 is captured by a signal receiver 20 that determines the coupling between the pair of PCBs 10. Based on previous calibrations, it is possible to determine the distance between the pair of PCBs after the tests based on the determined coupling. Since the distance before the tests is known, it is possible to determine the new position of the receiving PCB 10 and, consequently, the deformation suffered by the layer where the receiving PCB 10 is located and the contribution of this layer to the total deformation of the pavement. Furthermore, the use of the set of three coils allows an estimate of any horizontal displacements or rotations that may occur between the pavement layers.

### FIELD OF THE INVENTION

The present invention falls within the field of materials and civil engineering. More specifically, the present invention is related to a system and a method for separately assessing the deformation of each of the layers of a pavement.

### BACKGROUNDS OF THE INVENTION

Monitoring the contribution of each layer of a pavement to the total plastic deformation throughout the useful life of the structure is not trivial. On highways, monitoring this defect is done only superficially, observing the total deformation of the pavement without distinguishing the importance and contribution of each layer of the pavement to the deformation. In special cases, trenches can be opened transversely to the pavement in order to verify in situ the contribution of each layer, but in a very destructive and punctual manner, both spatially and temporally.

In instrumented experimental sections, greater emphasis is given to monitoring elastic stresses and deformations caused by the individual passage of each load, normally associated with the fatigue of the asphalt materials (Gonçalves, 2002; Colpo et al., 2020). When plastic deformations are considered, some alternatives can be used, among which there can be mentioned the use of LVDT (linear variable differential transducers), MDD (multi-depth deflectometers) and inductor coil cells, according to Dawson, 1994; Brito, 2011; Saevarsdottir et al., 2016; Greenslade, 2016; and Tian et al., 2017. Of these, the former are more complex and costly. In addition, their installation tends to be more invasive to the pavement, and can cause changes in the structure exactly at the point being monitored.

In turn, the inductor coils are simpler to manufacture. They operate in pairs, with a transmitter cell coil and a receiver cell coil. A signal with known frequency and amplitude is applied to the transmitter, inducing a magnetic field around the same. The oscillation of this magnetic field, in turn, induces a current in the receiver cell coil, which is translated into an output signal. The ratio between the output signal and the input signal is called coupling, which is maximum when the devices touch and decays as they are moved apart. By calibrating a pair of coils for different known distances, it is possible to infer unknown distances based on the observed coupling.

FIGS. 1 and 2 show cell coils according to the state of the art produced at UFRGS (Universidade Federal do Rio Grande do Sul). The multi-turn cell coils comprise coils of enameled copper wire with a diameter wound around a spool of polyacetal or other suitable material. The conventional cell coil is cheap and easy to manufacture, but it is unable to distinguish possible horizontal rotations and translations from vertical translations, since these movements will affect the system's single output signal in the same way-attenuating or intensifying the signal. In this way, its application in the field must be careful in order to maintain alignment between the pairs of cell coils, and even then, reading errors may occur.

Monitoring permanent deformations by means of surface measurements on the pavement is limited to assessing the pavement in an integrated manner, without observing the contribution of each layer to the total deformation. Therefore, given the need to better understand the influence of each pavement layer in relation to the permanent deformations, it becomes extremely important to instrument the experimental runways in order to measure the plastic deformations of each layer individually.

### STATE OF THE ART

Document RU189239U1, entitled “Probe for automated monitoring of residual deformation of multi-layered road constructions”, discloses a measuring probe consisting of a solid metal outer tube and a metal-plastic inner tube, with sensors fixed to it in a special frame, for example, precision analog Hall sensors. It also comprises an electronic system for measuring the accumulation of residual deformations in the layers of the road structure that operates according to the following principle: the magnetic system of two annular magnets enclosed in a hermetic casing has a section with magnetic induction varying linearly along the axis of the system. When moving the magnetic assembly in relation to the sensors, the induction of the magnetic field at the point of its installation changes and, consequently, changes the voltage at the output of the Hall sensor, which is recorded, for example, by a digital voltmeter. A measuring probe for automated monitoring of residual deformations of multilayer road structures is a measuring device placed in a pavement structure, consisting of an outer solid metal tube 2 and an inner metal-plastic tube 3, with measuring sensors 4 fixed on a special frame, 2 magnetic assemblies 5 and interlayer discs 6 are placed, the lower end of the device 7 has a pointed shape, freely immersed in the ground, flat at the lower end, combined with a pavement surface 8, screwed stopper 9 which is connected to the antenna terminal 10 of the radio module 11 transmitting information by radio to the measuring probe 12. The measuring probe is powered by a battery 13 located in the lower part of the multilayer tube 3. The interlayer metal discs 6 are made in a single design with a magnetic assembly 5 and are threaded into an outer metal tube 2. The number of discs 6 can be different and corresponds to the number of layers of the road structure 1 (if necessary, the interlayer discs 6 can be installed within the layers). The gap between the surface of the external metal tube 2 of the device and the internal surface of the magnetic assembly 5 is 1 mm; it cannot be smaller to allow the magnetic assembly 5 to slide along the device and it cannot be large to reduce the influence of the horizontal displacement of the assembly 5 in the magnetic field on the measurements. Inside the magnetic assembly 5 are magnetic rings made of an SmCo or NdFeB alloy.

The state of the art lacks systems and methods capable of determining the individual contribution of each of the pavement layers to the deformation. The state of the art also lacks systems and methods that achieve the above objectives without making the costs prohibitive.

### SUMMARY OF THE INVENTION

The device consists of three inductor cells mounted on a circuit board and positioned between pavement layers. Another device is positioned on the pavement surface after the pavement undergoes tension or stress. A known signal is fed to the surface device, which generates or induces a magnetic field in the inductor cells, which in turn is captured by the other device below the surface. The voltage induced in the device below the surface and the magnetic coupling between both devices are measured, which can determine whether and what type of deformation has occurred in the pavement. The use of three inductor cells allows the triangulation of the relative position of the sensors, thus mitigating possible misalignments, allowing a visualization of the movement of the cells on the three dimensional axes x, y and z—3D. With this, the proposed monitoring system aims at complementing the surface measurement campaigns planned to monitor the evolution of the permanent deformations on the experimental runways.

### BRIEF DESCRIPTION OF THE DRAWINGS

The present invention will be described below with reference to the typical embodiments of the same and also with reference to the attached drawings, in which:

FIG. 1 is a representation of a cell coil according to the state of the art;

FIG. 2 is another representation of a cell coil according to the state of the art;

FIG. 3 is a representation of the PCB of the system according to the present invention;

FIG. 4 is a detail of one of the PCB coils and its six circuit layers, according to the present invention;

FIG. 5 is a schematic representation of the positioning of the sensors;

FIG. 6 is a representation of the pavement deformation determination system according to the present invention;

FIG. 7 is a representation of the calibration curve of the pair of PCBs according to the present invention.

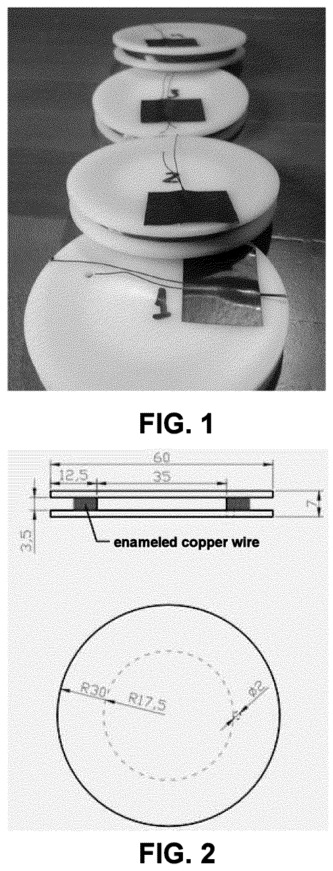

### DETAILED DESCRIPTION OF THE INVENTION

Specific embodiments of the present disclosure are described below. In an effort to provide a concise description of these embodiments, all features of an actual implementation may not be described in the specification. It should be appreciated that, in the development 41 any actual implementation, as in any engineering or design project, numerous implementation-specific decisions must be made to achieve the specific objectives of the developers, such as compliance with system-and business-related constraints, which may vary from one implementation to another. In addition, it should be appreciated that such a development effort may be complex and time-consuming, but would nevertheless be a routine design and manufacturing undertaking for those of ordinary skill having the benefit of this disclosure.

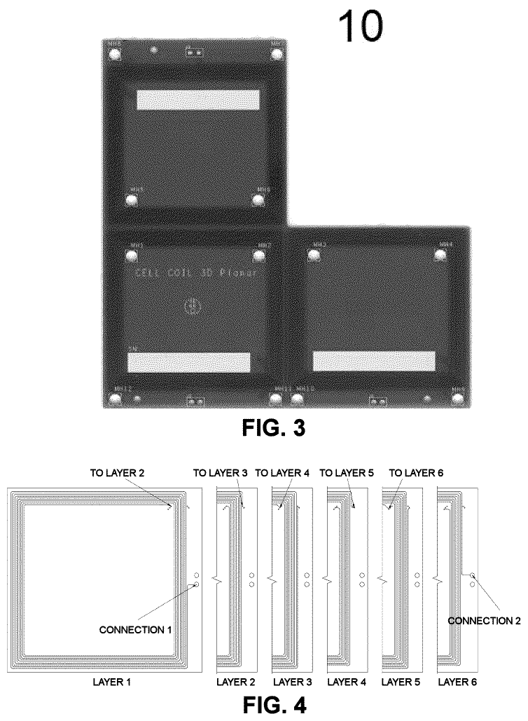

A first aspect of the present invention comprises a system consisting of two or more sets 10 of three inductor coils fabricated on printed circuit boards (PCBs). Similar to the cell coil of the state of the art, a pair of PCBs designed in this manner makes it possible to detect deformations by reading the magnetic field induced from the coils of a first PCB to the coils of the second PCB. The inventiveness of the present invention lies in the use of three coils instead of just one on each PCB. This allows the detection of misalignments and possible relative rotations between the PCBs by triangulating the signal in three dimensions-longitudinal, transverse and vertical. An illustrative and non-limiting representation of the set of coils mounted on a PCB is exemplified in FIG. 3. Each of the three coils of a PCB is printed in at least six layers of preferably ten turns each, without being limited to these values, the details of which are given in FIG. 4.

Since the main intention is to verify the reduction of the layer due to a deformation generated by the loading, one would expect there to always be an approximation of the plates (z axis). However, it should be noted that this may be accompanied by a planar movement caused by the deformation of the material (x, y). Thus, this invention aims at allowing the association of how much a possible planar separation that distances the sensors (since they are always positioned aligned during installation) is compensated by the vertical approximation (z axis) due to the permanent deformation. The multiple reading between the various sensors as transmitter-receiver pairs gives the proposed technique the ability to understand in a three-dimensional space the movement of each cell, thus ensuring more precise measurements of the monitored permanent deformation.

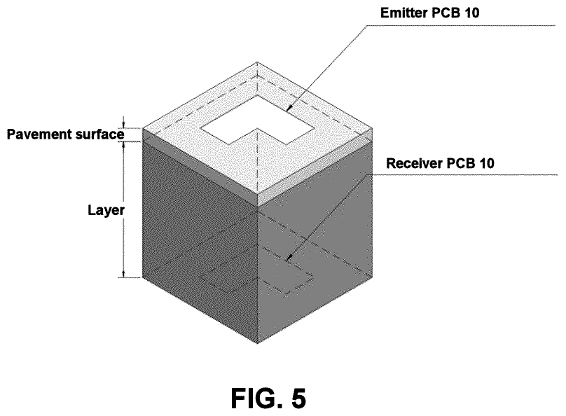

According to the present invention, a receiving PCB 10 is positioned between pavement layers of a pavement that will be tested. The receiving PCB 10 must be positioned directly on the compacted layer of interest during the preparation of the pavement. In this way, an interference with the pavement after its completion is avoided, ensuring the quality of the pavement, preservation of the position of the receiving PCB 10 and absence of deformations in the pavement. FIG. 5 illustrates the positioning of the receiving PCB 10 during the production of the pavement to be tested. The position of the receiving PCB 10 and its consequent distance from the finished pavement surface are taken at this point as they will be important during the tests. The outputs of the receiving PCB 10 are wired to a signal receiving device, which can be any device of the state of the art suitable for this purpose.

During the preparation of the test pavement, multiple receiving PCBs 10 may be positioned in any of the compacted layers. Preferably, they will be spread in different layers and separated by a sufficient horizontal distance to avoid an interference between receiving PCBs 10. The installation of multiple PCBs 10 at the same point at different depths is possible, provided that an adjustment factor is applied to the air-calibrated curve (FIG. 4) to compensate for this and other field factors (soil type, moisture).

After the test pavement is completed and the test procedures are performed, an emitting PCB 10 is positioned on the pavement surface so as to align vertically with one of the receiving PCBs 10 positioned in one of the layers below, forming a pair of PCBs 10 (FIG. 5).

The emitting PCB 10 is connected to a signal generating device as known in the art to excite the coils of the emitting PCB 10 which, in turn, excites the coils of the receiving PCB 10. The signal received by the coils of the receiving PCB 10 is transmitted to the signal receiving device.

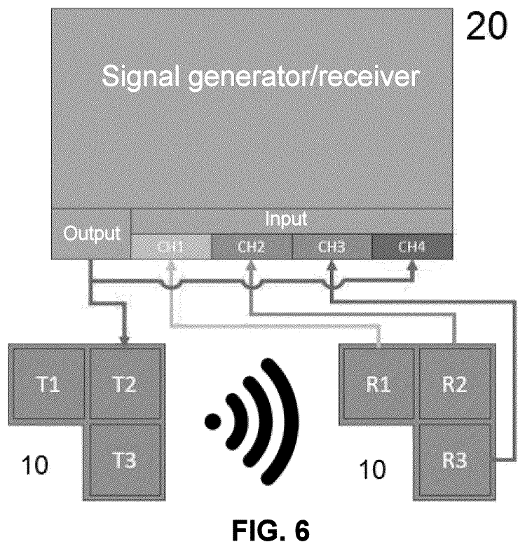

The resulting system is exemplified in FIG. 6. For validation of the system of the present invention, a single device 20 acts as both signal generator and receiver. A Keysight InfiniiVision 3000T X-Series oscilloscope was used to generate signals to be supplied to the emitting PCB 10 and to receive the signals transmitted by the receiving PCB 10. It is evidently possible to use separate devices to generate and receive the signals. For simplicity and convenience, the description that follows assumes the use of a single device 20.

The signal generator/receiver 20 transmits an emitted signal to one of the coils of the PCB 10 (T1, T2 or T3), inducing a magnetic field. For example, the emitting signal used in the validation was a sinusoidal signal with a frequency of 520 MHz and an amplitude of 5 V. This induced magnetic field is, in turn, captured by the coils R1, R2 and R3 of the receiving PCB 10 positioned between the layers of the pavement, generating a resulting signal that is related to the original signal emitted by the signal generator/receiver 20. The resulting signal is captured by the signal generator/receiver 20 by connecting each coil R1, R2 and R3 to one of its reception channels, for example, the channels 1, 2 and 3 of the oscilloscope, CH1, CH2 and CH3, respectively. Preferably, to validate the signal effectively emitted by the signal generator/receiver 20 to the emitting PCB 10, a signal splitter can be used by connecting the signal generator/receiver 20 to a fourth channel CH4 of the data acquisition. The process can be performed three times, once for each transmitter (T1, T2 and T3), allowing for a better estimate of the horizontal displacements.

As is known, the relation between the emitted signal and the resulting signal is called coupling and is defined as the ratio between the emitted signal and the resulting signal. The coupling is maximum, i.e. equal to 1 or 100%, when the distance between the emitting device and the receiving device is minimum, and decreases as they are moved apart, tending to zero or 0%. By calibrating a pair of emitting/receiving devices for different known distances, it is possible to infer unknown distances based on the observed coupling.

It is important to note that, depending on the type of soil and other factors, such as soil moisture, an adjustment factor for the air-calibrated curve may be necessary for compensation.

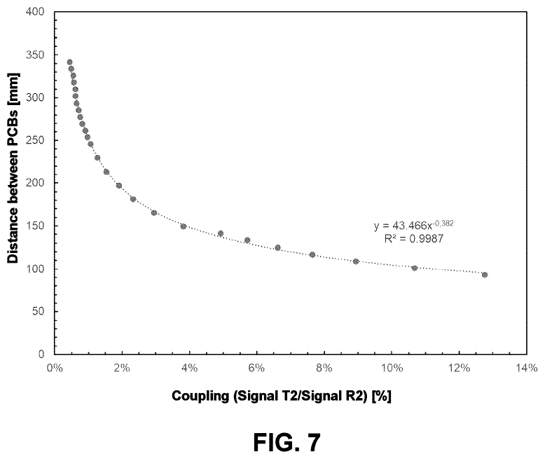

When validating the invention, a pair of PCBs 10 was calibrated for a response dimension (i.e., perfectly aligned sensors, without considering horizontal displacements and varying only the linear distance between PCBs), in open air. FIG. 7 presents the results obtained when the coil T2 is directly excited by the signal generator and the response is read from the coil R2. By means of linear regression, an approximate curve of the type y=ax^{b} with coefficient of determination R²=0.9987 was obtained. The calibration thus obtained is summarized in Equation 1:

$$distance [ mm ] = 43.466 \cdot {coupling}^{- 0.382} \qquad ( 1 )$$

Coupling is defined as in Equation 2:

$$coupling = \frac{voltage received at R 2 [ V ]}{voltage transmitted by T 2 [ V ]} \qquad ( 2 )$$

The calibration considering other degrees of freedom (transverse, longitudinal displacements and, eventually, rotations between PCBs) is more complex and requires the joint analysis of the three response signals simultaneously (R1, R2 and R3). The technician skilled on the subject will be able to obtain the necessary calibration for specific applications without departing from the scope of the present invention.

The coupling curve for each pair of emitting PCB 10 and receiving PCB 10 is obtained and also the initial coupling between each pair before the pavement is tested. After the tests, the new coupling between each pair is determined. Based on the new coupling, it is possible to determine the new distance between the pair and, consequently, the new position of the receiving PCB 10. This allows the determination of the plastic deformation suffered by the pavement layer where the receiving PCB 10 is located and also the contribution of this layer to the total deformation of the pavement.

A second aspect of the invention is related to a method of testing pavements using the system defined in the first aspect of the invention. The method comprises the steps of:
Positioning one or more PCBs 10 between layers of a pavement to be tested, preferably during the manufacture of the pavement to ensure the quality of the same, in which the one or more PCBs 10 will be receiving PCBs 10 and are connected to a signal receiver 20;
Performing tests after the pavement is completed;
Positioning another PCB 10 on the pavement surface so that it is aligned in the height direction with one of the receiving PCBs 10 forming a pair of PCBs 10, in which the other PCB 10 will be an emitting PCB 10 and is connected to a signal emitter 20;
Emitting a signal emitted from the signal emitter 20 to the emitting PCB 10 and receiving a resulting signal from a receiving PCB 10; and
Determining a coupling between the pair of PCBs 10, in which the coupling indicates a distance between the pair of PCBs 10.

Advantageously, the techniques described herein do not require the opening of trenches and are not limited to a relatively small area of the pavement being tested.

The system and method described herein allow the assessment of the permanent deformation of pavement layers in situ, enabling the obtaining of wheel track data from experimental runways for analyses of permanent deformation in pavements, enabling the calibration of the shifting model for both internal and external analyses. The present invention will allow the best choice of pavement materials, reducing costs and increasing the durability of pavements.

The present invention is advantageous in relation to alternative methodologies because it is low cost and in 3D.

Although aspects of the present disclosure may be susceptible to various modifications and alternative forms, specific embodiments have been shown by way of example in the drawings and have been described in detail in this document. But it should be understood that the invention is not intended to be limited to the particular forms disclosed. Rather, the invention is intended to cover all modifications, equivalents, and alternatives that fall within the scope of the invention as defined by the following appended claims.

### REFERENCES

Brito, L. A. T. (2011). Design Methods for Low Volume Roads. Thesis (PhD). University of Nottingham.
Colpo, G. B. et al. (2020). Fatigue behavior study of a dense graded HMA using the four-point bending beam test aided by an in-situ instrumentation at BR-116/RS, Brazil. Revista Transportes. V. 28, n. 2.
Dawson, A., The ε-mu System, User's Manual, 2^{nd} edition, 1994 (University of Nottingham: Nottingham, UK). Gonçalves, F. J. P. (2002) Estudo do Desempenho de Pavimentos Flexíveis a partir de Instrumentação e Ensaios Acelerados. Thesis (Doctorate in Engineering). Universidade Federal do Rio Grande do Sul, Porto Alegre.
Greenslade, F. R. (2016). Development of a New Pavement Strain Coil Measuring System at CAPTIF.
Saevarsdottir, T. et al. (2015). Instrumentation and performance modelling of heavy vehicle simulator tests. International Journal of Pavement Engineering. V. 17, n. 2. P. 148-165.
Tian, Y. et al. (2017). Development of a mid-depth profile monitoring system for accelerated pavement testing. Construction and Building Materials. V. 140, p. 1-9.

1. A system for assessing pavement deformation, comprising:
an emitting PCB and at least one receiving PCB, each of the emitting PCB and the at least one receiving PCB comprising three coils installed therein, wherein the emitting PCB forms a pair of PCBs with each of the at least one receiving PCB;
a signal generator configured to emit a signal emitted to the emitting PCB; and
a signal receiver configured to receive a resulting signal from the at least one receiving PCB;
wherein the signal receiver is configured to determine a coupling between the pair of PCBs, the coupling being an indicator of the distance between the pair of PCBs.

2. The system according to claim 1, wherein the signal generator has a signal splitter so that the emitted signal is also supplied to the signal receiver.

3. A method of using the system to assess pavement deformation defined in claim 1, comprising the steps of:
positioning the at least one receiving PCB in a layer of a pavement;
performing tests on the pavement after it is completed;
positioning the emitting PCB on the pavement surface so as to align the pair of PCBs in the height direction;
emitting the signal emitted from the signal generator and receiving the resulting signal in the signal receiver; and
determining the coupling of the pair of PCBs, the coupling being indicative of a distance between the pair of PCBs.
